(19) United States
(12) Patent Application Publication
(10) Pub. No.: US 2010/0029261 A1
(43) Pub. Date: Feb. 4, 2010

(54) Virtual wireless data cable method, apparatus and system

(76) Inventors: John Mikkelsen, Minneapolis, MN (US); Robert Freidson, St. Petersburg (RU)

Correspondence Address: John P. Luther;Ladas & Parry LLP, Suite 1600, 224 South Michigan Ave., Chicago, IL, 60604, US

(21) Appl. No.: 12/322,609

(22) Filed: Feb. 4, 2009

Related U.S. Application Data
(63) Continuation-in-part of application No. 10/183,756, filed on Jun. 26, 2002, now Pat. No. 7,548,875.
(60) Provisional application No. 60/343,159, filed on Oct. 26, 2001.
(60) Provisional application No. 60/312,450, filed on Aug. 14, 2001.
(60) Provisional application No. 60/303,115, filed on Jul. 3, 2001.
(60) Provisional application No. 60/301,681, filed on Jun. 27, 2001.

(51) Int. Cl. H04M 3/00; G01M 17/007; G06F 17/00; G06Q 30/00; G10L 15/00; A61B 5/00

(52) U.S. Cl. 455/419; 701/33; 701/36; 705/26; 704/275; 600/300; 704/E15.001

(57) ABSTRACT

A system, method and apparatus to establish a complete and symmetrical virtual wireless cable for data transmission in such a way that devices, attached to both ends of the system, could use it as if it is a simple regular data cable.

### RELATED APPLICATIONS

This application is a Continuation-in-Part of U.S. Ser. No. 10/183,756, filed Jun. 6, 2002, now U.S. Pat. No. ______, which claims priority to U.S. Provisional Application Ser. No. 60/303,115, filed Jul. 3, 2001, U.S. Provisional Application Ser. No. 60/312,450, filed Aug. 14, 2001, and U.S. Provisional Application No. 60/343,159, filed Oct. 26, 2001, all of which applications are incorporated by herein reference

### FIELD OF THE INVENTION

This invention relates to a method, system and products for the transmission, delivery, playback, and content management of audio and visual files for wireless and non-wireless devices, and a new Internet-less protocol for such transmission to portable electronic devices, such as cell phones and the like, which essentially eliminates the need for conventional cable connections.

### BACKGROUND OF THE INVENTION

Serial cables, which use RS-232 standards for their connectors, are commonly used to transfer information between two or more devices, such as communicating with a computer and a peripheral, for example, a modem. Other devices which commonly communicate via serial cable include, for example, printers, personal digital assistants, medical devices, gaming devices, barcode scanners, cash drawers, magnetic strip readers, amateur electronics, remote-control devices, field equipment, and various military equipment. While the technology is not current, serial cables may still be found in wide use in electronic devices that do not require high bandwidth communication. Other severe limitations of serial cable employing RS-232 standards for their connectors is a cable length which must be decreased or sacrificed for the sake of communication speed, which is slow in any event (up to 115,200 bits per second, or otherwise referred to as “bits/s” or “baud”) by today's bandwidth requirements which can exceed 1,500,000 baud. Reducing speed by a factor of from, say, 2-4 will allow maximum cable length to be increased, for example, up to about fifty feet, which is unsuitable for many of today's uses.

Recently, growing use of other communication cables, such as those needed for USB and FireWire connections, have replaced the serial RS-232 standards cable in many applications, especially for local communications. For example, USB provides for faster communication, uses lower voltage levels and is seen as having connectors which are simpler to connect and use. On the other hand, USB is more complex than the RS-232 standard and requires more software to support the protocol used. Serial ports of personal computers were also often used to directly control hardware devices, such as relays, as interface control lines could be conveniently manipulated by software. This cannot be done with USB, however, which requires a form of receiver to decode serial data.

USB cable is also limited by maximum length, which is slightly more than fifteen feet, and unacceptable for many applications. Additionally, if a USB device does not answer to host commands within an allotted time, the host considers the command to be lost, which is seen as a potentially severe limitation as interrupting traffic and requiring human intervention. Using USB cable over a greater length requires additional equipment in the form of hubs or active extension cables and other equipment, which, of course, can dramatically increase installation and operating expense, and making many routine applications prohibitively expensive for common consumers. Furthermore, a tangled web of unsightly cables has plagued consumers for years.

The present inventive methods, apparatus and system completely solves all of the problems with conventional cable technology, and provides for such advantages as a fast and convenient wireless and cable-less connection and operation of an essentially limitless array of electronic devices over any distance, or within any compact area, without a web of cables, and which will correct itself automatically without the need for human intervention.

The present invention is more fully described with reference to the following detailed description of preferred embodiments with accompanying drawings.

### SUMMARY OF THE INVENTION

The present invention is a system, method and apparatus for providing essentially complete and symmetrical virtual wireless cable for data transmissions (hereinafter “W-Cable tm”) which enables one or more or a plurality of electronic devices attached or otherwise virtually connected to terminal ends of the W-Cable for utilizing the inventive W-cable in the same manner as conventional cables.

In a preferred embodiment, the inventive W-Cable may comprise several conventional devices and several novel devices as shown in FIG. 1, which is discussed more fully below. By way of the W-cable method and apparatus there is now provided a new and novel system which can be utilized in a variety of applications, which at present either use common RS-232 or USB interfaces or employ other interfaces using custom-made adapters, and provides several advantages over conventional wireless radio channel methods of data transmission.

In one aspect of the invention, the W-Cable may be inserted or otherwise installed between one or more or a plurality of devices which have heretofore been connected via conventional RS-232 (or USB) cable. No modification of any device is required, nor is there a need to employ any specific function to control wireless channels, such as calling a remote party, monitoring a line condition, setting up connections, enabling error correction, re-connecting when a connection is lost, transferring states of control lines, etc. Additionally, computer or Internet access is not required on any terminus of the W-Cable. The only requirement is the W-Cable itself, and in which all operations thereof are completely transparent for one or more or a plurality of devices used therewith.

The inventive W-Cable may support full 9-pin RS-232 interface for any direction completely symmetrically. In contrast to conventional devices for similar usage, the W-Cable transmits all control signals of RS-232-9 in addition to data transmit/receive channels, and which may be extremely advantageous for interconnection of devices that use control signals (such as DTR, CTS, CD, DSR, etc.), for instance, for data flow control.

In a preferred embodiments, the W-Cable provides for a virtually continuous connection. Restoring a connection after link failures, transmit errors, data re-transmission, flow control, synchronization of data rates, data buffering, when and if necessary, may be accomplished via the W-Cable and external devices such as simple flow control. For example, in the instance that a connection is broken an external device receives notification that a buffer is full and in accordance with the invention will suspend data transmission, in contrast to conventional systems which would send a “connection is lost” signal, or similar signal, which would interrupt the normal function of the device and, in many cases, would require human personnel intervention to “reset” the device or otherwise restore functionality.

Further, unlike any other conventional methodology and products, the inventive W-Cable advantageously employs cellular telephony system for data transmission, which provides for high connection availability and reliability essentially everywhere in any developed country, or substantially worldwide, and combined with a reasonable cost and which eliminates the need of oftentimes troublesome antennae or power sources, radio link licensing, and provides for high data transmission rates—9600 bit/s as a minimum. An additional advantage is that operational service may be conveniently charged to a cellular provider, which may be substantially less than the cost of buying or leasing other sorts of radio equipment.

In further advantageous respects the W-Cable does not require the use of satellite communications, which reduces cost requirements and enables the use of conventionally small equipment dimensions. For example, the mobile portion of the W-Cable may easily fit into a ladies handbag or smaller compartment. At any time, one may use a phone to make a call to supply additional information.

Further advantageous, is the W-Cable does not require an Internet connection or services, computers, leased lines or any other special equipment. An ISP connection is simply not needed. Only a simple phone line and the second portion of the W-Cable, which does not exceed the mobile counterpart in size, are employed as basic components all of which greatly reduces the cost of production and sales price.

The inventive apparatus and method may be implemented with both RS 232 data transmission protocol and USB transmission protocol (USB wireless cable).

### BRIEF DESCRIPTION OF THE DRAWINGS

FIG. 1. is a schematic diagram of a preferred embodiment of a virtual wireless cable system in accordance with the present invention.

FIG. 2. is a schematic diagram of a preferred embodiment of a board system implemented in a Mobile Intellectual Unit in accordance with the present invention.

FIG. 3. illustrates a block diagram of a preferred embodiment of a wireless system in accordance with the present invention.

FIG. 4. illustrates a block diagram of a conventional landline system in accordance with a preferred embodiment of the present invention.

FIG. 5. illustrates a block diagram of a preferred embodiment of a remote control system in accordance with the present invention.

FIG. 6. illustrates a block diagram of a preferred embodiment of a home control system in accordance with the present invention.

FIG. 7 illustrates in preferred embodiment a mock up of a human body with remote monitoring of an array of diagnostic indicators in accordance with the invention.

FIG. 8 illustrates in preferred embodiment a transparent mock up of an automobile with remote monitoring of an array of diagnostic functions in accordance with the invention.

FIG. 9 illustrates in preferred embodiment a home environment with remote control and/or monitoring of an array of functions in accordance with the invention.

### DETAILED DESCRIPTION OF PREFERRED EMBODIMENTS

All patent references, published patent applications and literature references referred to or cited herein are expressly incorporated by reference to the same extent as if each were specifically and individually indicated to be incorporated by reference. Any inconsistency between these publications and the present disclosure is intended to and shall be resolved in favor of the present disclosure.

In the following discussion, many specific details are provided to set forth a thou rough understanding of the present invention. It will be obvious, however, to those skilled in the art that the present invention may be practiced without specific details, and in some instances of this discussion with reference to the drawings known elements have not been illustrated in order not to obscure the present invention in unnecessary detail. Such details concerning computer networking, software programming, telecommunications, and the like have not been specifically illustrated as such are not considered necessary to obtain a complete understanding of the core present invention, but are considered present nevertheless as such are considered to be within the skills of persons of ordinary skill in the art.

It is also noted that, unless indicated otherwise, all functions described herein may be performed in either hardware or software, or some combination thereof. In some preferred embodiments, the functions are performed by a processor such as a computer or an electronic data processor in accordance with code, such as a computer program code, software, and/or integrated circuits that are coded to perform such functions.

Additionally, the processing that is depicted n the drawings and described below is generally depicted as hierarchical structure for readability and understandability. Various other methodologies, such as object-oriented techniques, are preferred for the physical embodiments of the invention in order to maximize the use of existing programming technique. One of ordinary skill in the art will appreciate that the techniques described herein may be embodied in many different forms.

For illustrative purposes only, the following discussion illustrates and discusses the present invention in reference to various embodiments which may perhaps be best utilized subject to the desires and subjective preferences of various users.

Having thus prefaced this discussion, the inventive apparatus and method is based on radio-frequency (RF) remote control which eliminates the need for length-constrained physical cables and provides distinct advantages over light remote controlled devices, such as infrared control. As known, instead of sending out light signals, on RF remote transmits radio waves that correspond to the binary command for an executable function, such as a pushed button. A radio receiver on a controlled device receives the signal and decodes it. Interference problems due to the large amount of radio signals present at any given time are usually handled by transmitting at specific radio frequencies and by embedding digital address codes in the radio signal. This capability lets the radio receiver on the intended device know when to respond to a signal and when to ignore it.

RF is any frequency within the electromagnetic spectrum associated with radio wave propagation. When RF propagation is supplied, an electromagnetic field is created that is then able to propagate through space. As known there are many wireless technologies that are based on RF field propagation.

In accordance with the present invention there is provided a virtual wireless cable system employing RF for data transmission for controlling a plurality of digital data signals transmitted to one or more remotely located devices operatively effective to carry out controllable function upon receipt of a command delivered by a control signal from a first device. The transmission operation comprises delivering over the air wirelessly a digital audio and/or visual content signal from the first device to a remotely located device in compressed and optimized format wherein the digital audio and/or visual content signal is compressed and optimized by one or more algorithms effective for compression and optimization of said content for over the air wireless delivery.

The present inventive method, apparatus and system is preferably enabled and operated in accordance with that described by the principles set forth in full in U.S. Published patent application Ser. No. 10/183,756 for a Media Delivery Platform and U.S. patent application Ser. No. ______ for an Improved Media Delivery Platform, both of which are incorporated by reference herein in their entirety.

In these novel methods, both audio and visual content in digital form is transferred wirelessly over-the-air (OTA) without the need for an Internet or ISP connection (although an Internet connection may also be employed) in algorithmically compressed and optimized format to any of an array of electronic receiver devices on demand for operation of a limitless array of functions, such as providing any kind of information in textual or visual form or a combination thereof, commands to operate machinery, electronic motors for appliances, automotive and other vehicle and watercraft ignition, diagnostic devices to harvest human health and mechanical performance information and the like, movies, or other rich media, such as a library of CD quality master tones as desired. A preferred electronic device for use may be a cell phone or a device with cell phone capability, or a landline device. Thus, with the use of this inventive technology wireless high speed information or commands may be transmitted as desired, or harvested as desired, such as in the case of provided or generated diagnostic information, in virtually any application or confined space or long-range over large distances, such as the entirety of an industrial complex or from a home environment to a business environment located miles away.

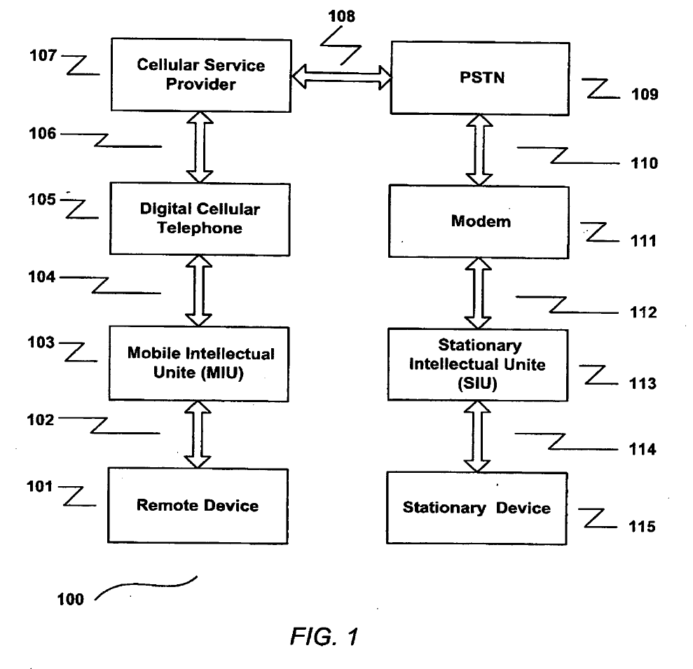

Turning now to FIG. 1, in a preferred embodiment system 100 comprises an asymmetric structure with Mobile Component 101 and the Stationary Component 115. Alternatively, there may also be provided a symmetric system with two mobile terminations wherein both ends are mobile. A Mobile Intellectual Unit (MIU) 103 receives data from the Remote Device 101, buffers it, provides continuousness of connection with the opposite end of the W-Cable, corrects data transmission errors and implements a protocol to control and transmit states of the control lines. The MIU 103 is furnished with a power supply and cables for connection to cellular phone 105.

Mobile Component 101 preferably comprises two MIUs 103 and a Digital Cellular Phone 105 (for example a 2.5 or 3G GSM Cellular Phone). The cellular phone 105 is used for data transmission (GSM standard supports at least 9.6 Kbit/s data rate, with possible improvements in new standards like 3G Up to 1,500 Kbit/s).

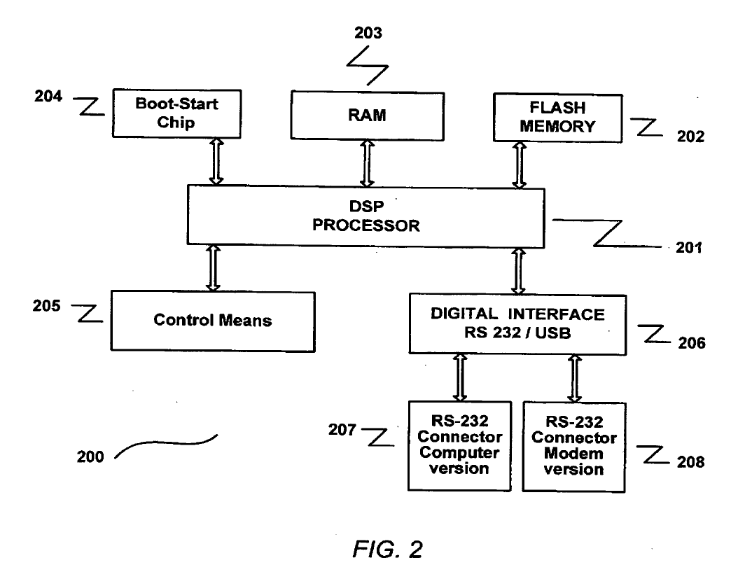

A preferred embodiment of a schematic diagram of a board system implemented in a MIU is shown in FIG. 2. The MIU 200 carries out all the functions that are required to create virtually error-free, continuous and buffered channel out of an unreliable and inconvenient channel provided by the phone. In this preferred embodiment the MIU 200 comprises DSP Processor 201, Flash memory 202, Random Access Memory (RAM) 203, Boot Start Chip 204, Control and State Indication capability 205 and Digital Interface 206, with two RS-232 connectors: 1) a computer version 207 (Tx direction: Data, Dtr. RTs; Rx direction: Data, Cts, Dsr, Ring, Cd) and 2) a modem version 208, where control signals have opposite directions. Control 205 allow the selection of data rate, flow control type, and other parameters, so that the MIU agrees with, i.e. is in sync with, the external device 101.

Referring back to FIG. 1, a Stationary Component 115 comprises a Stationary Intellectual Unit (SIU) 113, similar in functionality to the MIU 103, and a conventional external Modem 111. The SIU differs from the MIU in that the SIU controls a modem and not a cellular phone. From the user point of view, the SIU 113 has the same connectors, controls and indication capability as the MIU, as shown in FIG. 2. the preferred embodiment of a schematic diagram of a board system implemented in a MIU. The SIU is furnished with a power supply and cables for connection to the modem.

In further preferred and advantageous embodiments, the inventive system may be operational as packet switched, such as commonly used in Internet applications, but operational over a PSTN which eliminates the need for an Internet or ISP connection, but which can also employ an Internet connection.

In an active mode of data transmission Remote Device 101 transmits data packets to the MIU 103 on the mobile termination. The MIU 103 receives the data and stores it in its RAM buffer 203. The MIU 103 also receives and records states of all control signals of RS-23 presented to a Stationary Device 115. Then the MIU 103 uses the cellular telephone 105 to connect to a Stationary Device 115. If the number is busy or a failure occurs, the MIU 103 automatically retries until a connection is established. The SIU 113 then connects to the remote portion of the system using the following path: Remote Device 101→MIU 103→Cell. Telephone 105→Cell. Service Provider 107→PSTN 109→Modem 111→SIU 113→Stationary Device 115.

If a failure or disconnection occurs during the data transmission, the MIU 103 automatically re-establishes the connection and resumes data transmission, unlike a failed transmission via a standard USB cable. Finally, when all information is in the memory of the SIU 13, the SIU will output data through its local RS-232 interface to the Stationary Device 115, in the same manner as if a Remote Device 101 was connected to a Stationary Device 115 through a simple conventional RS-232 cable. The SIU 113 will also set RS-232 control signals as if they were driven by a Remote Device 101.

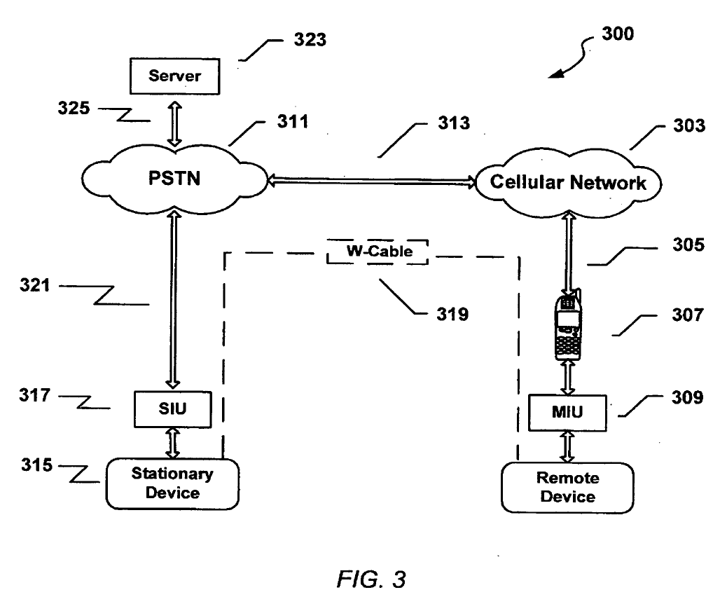

FIG. 3 illustrates a preferred embodiment via a block diagram of a wireless system in accordance with the present invention. Included in the system 300 is an operational cellular network 303 in wireless connection via wireless link 305 with one or more cell phone units 307 comprising a Mobile Intellectual Unit (MIU) 309 (or a Remote Intellectual Unit—RIU 309). A Public Switched Telephone Network (PSTN) 311 is in wireless communication via wireless link 313 with the cellular network 303. PSTN 311 is also in communication via landline link 313 with one or more stationary devices 315, such as a service or control object for generating diagnostic information, comprising a Stationary Intellectual Unit (SIU) 317. Both of MIU 309 and SIU 317 are in communication with PSTN 311 and the cellular network 303 via W-cable 319, and PSTN 311 may be connected by a landline link 321, or any suitable link, to one or more servers (e.g. “Neutral Net Environment Virtual Agent” as shown), by way of an Internet link, or Internet Service Provider (ISP) to an Internet accessible site.

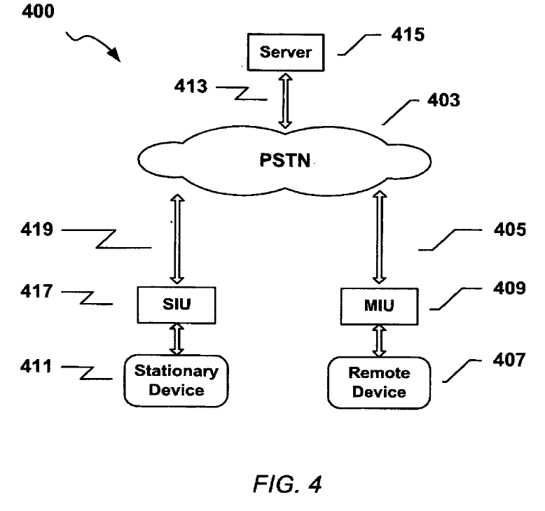

FIG. 4 illustrates in a preferred embodiment by way of a block diagram, a conventional landline system 400 for use in the present inventive method, apparatus and system. As shown, PSTN 403 is in landline communication via link 405 with one or more remote devices 407, such as a conventional landline phone, which comprises Remote Intellectual Unit (RIU) 409. PSTN 403 is also in landline communication via link 409 with one or more service or control objects 411, such as a diagnostic unit. PSTN 403 is in further communication via landline link 413 to one or more servers 415 (referred to as “Neutral Net Environment Virtual Agent” as shown) which is connected via an ISP 417 to an Internet accessible web site.

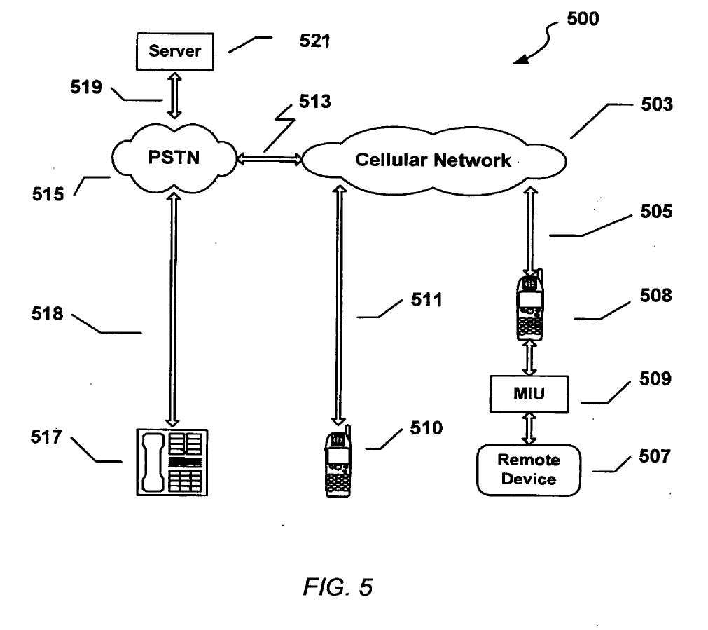

A preferred embodiment, shown via a block diagram, of a remote control system in accordance with the invention is set forth in FIG. 5. In this system 500 cellular network 503 is in communication via wireless link 505 with one or more remote control objects 507, such as a cell phone or a remote control device situated in an auto or home, and which comprises RIU 507. Cellular network 503 is accessible for receiving commands or otherwise contact from cell phone 507 via wireless link 511. Cellular network 503 is in communication via wireless link 513 with PSTN 515, which, as shown, may also be in communication with one or more landline home telephones 517. PSTN is linked via landline link 519 to one or more servers 521 (referred to as Virtual Intelligent Agent With Voice Recognition” as shown) which is connected via ISP 523 to an Internet accessible web site.

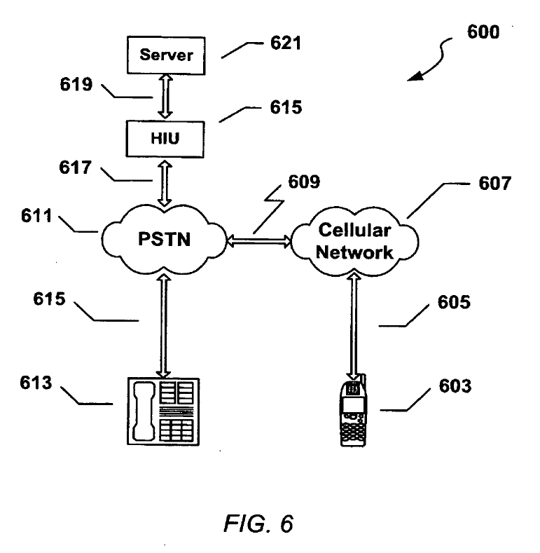

In FIG. 6 there is illustrated in block diagram a preferred home control system in accordance with the invention. In system 600 one or more cell phones 603 is in wireless communication via wireless link 605 with cellular network 607, which in turn is in wireless communication via wireless link 609 with PSTN 611. One or more home telephones 613 may be in communication via a landline link 615 with PSTN 611. Further, PSTN 611 is in communication with Home Intellectual Unit(s) (HIU) 615 via a landline link 617 which in turn is in communication via landline link 619 with one or more servers 621 (referred to as “Virtual Intelligence Agent With Voice Recognition” as shown) which is connected via ISP 623 to an Internet accessible web site.

There are obviously many preferred applications for the inventive methods, apparatus and system, with such applications only limited by one's imagination. For example, a major advantage of application of the W-Cable system, such as system 100 shown in FIG. 1, is the W-Cable itself which comprises within all needed operational units and protocols, and is completely transparent. This important property allows the connection via W-Cable of essentially any and all remote devices which do not have pre-existing tools, such as Windows or the Internet, but only a simple standard digital interface and which devices cannot themselves perform any functions for error correction, connection establishment or recovery after a failure. It will be appreciated that such a remote connection would not be possible without the employ of the inventive W-Cable, or some conventional analogue, usually more bulky and expensive, such as a set of Windows computers and non-trivial software.

As mentioned above, there exists a virtually limitless spectrum of existing special-purpose devices which may be connected and/or operated by the inventive W-Cable system, including but not limited to, applications in transportation and traffic control (vessels, airplanes, cars); security and terrorist combating systems; power systems; banking system equipment; distributive trade; applications in the entertainment industry; home use equipment and many, many more, as only limited by the imagination.

In an example of law enforcement and government over-the-air (“OTA”) transmission application, a cell phone may be used as a personal ID to provide for access to secure areas, such as in train stations, airports, stadiums and the like. Via a cell phone and the inventive W-cable, a biometrics application my be employed to transmit one's “fingerprint” to a control terminal which may be connected to a government database, or world wide database, such as Interpol, and permit access to a secure area or secured information.

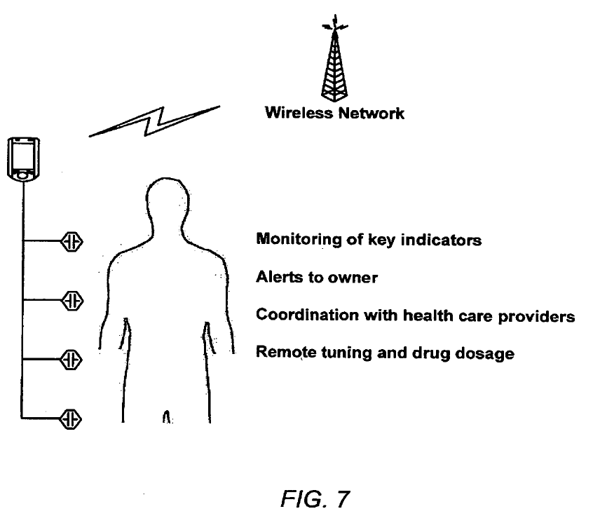

In another example, as shown in FIG. 7, a wireless OTA transmission may be accomplished via a cell phone enabled with attachments to provide an efficient remote diagnostic tool, for instance, to monitor blood pressure, insulin levels, cardiograph indications and the like. If required, there may be a continuous monitoring of key indicators with alerts provided to a user or other individual, and with essentially instantaneous coordination with health care providers, and other advantages, such as remote tuning of drug dosage regimens.

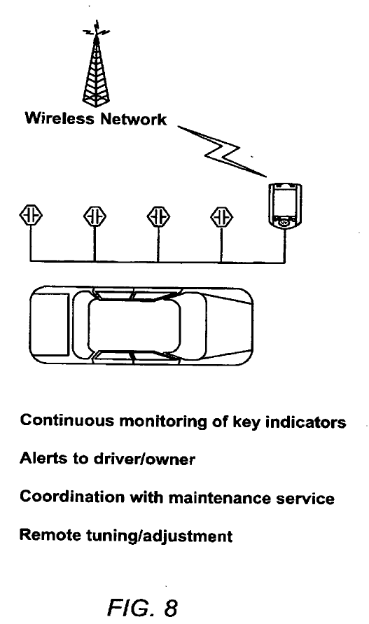

In other examples, as shown in FIG. 8, an automobile diagnostics system may be provided, wherein an adapter translates data from some proprietary automobile interface into RS-232, followed by data transmission via the W-cable to the stationary side, and followed by a second adapter transferring data into the testing equipment using its proprietary interface. The efficiency and advantage of such an application is provided by its relatively low cost, high reliability and the small dimensions and weight of the W-Cable system. At the same time, a user would always have the capability of making a conventional voice phone call using the same cell phone. As set out in FIG. 8, with the use of W-cable, it becomes a simple task to enable a complete remote diagnosis capability of one's vehicle, with the possibility of continuous monitoring of key indicators, such as brake condition, oil pressure/level condition, engine temperature, interior temperature, window and door locks, and essentially any function associated with vehicle operation and maintenance with essentially instantaneous coordination with a maintenance service department, and the capability of remote tuning and adjustments.

Additionally, with the use of the present invention, one may simply call their automobile or other vehicle or watercraft and the like from a remote location such as an office and start up the ignition, air conditioning, heater, radio or virtually any other function.

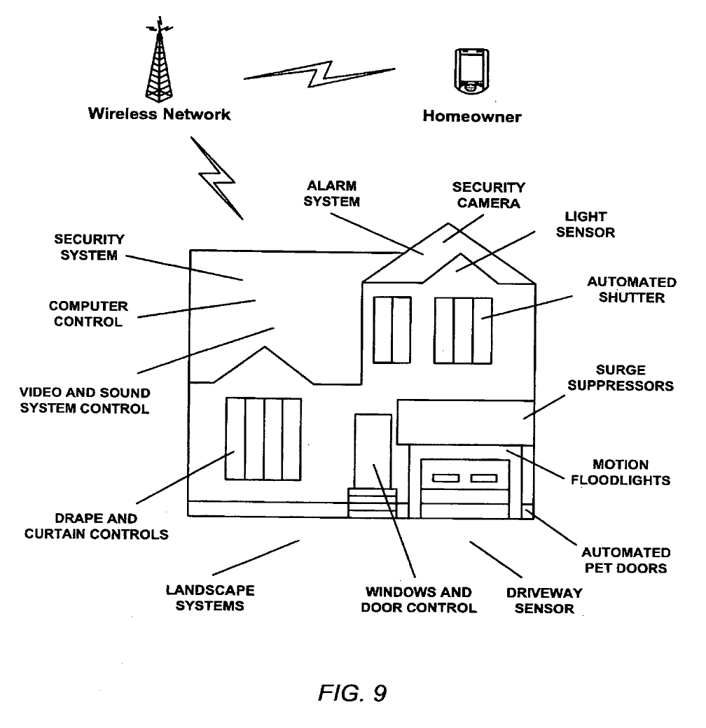

As shown in FIG. 9, there is illustrated in a preferred embodiment an array of some of the many functions and units which may be remotely monitored or controlled in accordance with the present invention without the use of cumbersome and length-limited conventional cable technology. As shown, these may include, but not limited to, window and door controls, telephone systems, computer control, security cameras, security systems, intercom systems, driveway sensors, mailbox sensors, automated shutter, drape and curtain controls, automated pet doors and pet feeders, surge suppressors, lighting control, motion floodlights, automatic sprinkling systems and landscape systems, such as automatic pond feeders, video and sound system control, in addition to (not shown) TVs, microwaves, refrigerators, garage doors and the like, all of which may be operated via internal micro controllers by way of a simple telephone call in accordance with the present invention.

Many additional applications exist, of course, including, for example, a barcode system to swipe and scan electronic barcode products to substantially instantaneously produce an order. This may be accomplished, for example, by using a conventional bar code reader to place an order at a remote location by way of a remotely controlled device.

In contrast to the Internet and conventional cable technology, requirements of the inventive W-Cable to produce a quality connection are rather low, as enabled via error correcting properties of the W-Cable, and in contrast to USB cable technology, channels always remain operational. The W-Cable is “patient and not forgetful”, even if the connection to the base station is lost; the W-Cable would wait until it resumes, and sooner or later, it would transmit all data and commands as intended automatically.

Although illustrative embodiments of the present invention have been described herein with reference to the accompanying drawings, it is to be understood that the invention is not limited to those precise embodiments, and that various other changes and modifications may be affected therein by one skilled in the art without departing from the scope or spirit of the invention.

1. A virtual wireless cable system for data transmission for controlling a plurality of digital data signals transmitted to one or more remotely located devices operatively effective to carry out controllable function upon receipt of a command delivered by a control signal from a first device, wherein said transmission comprises delivering over the air wirelessly a digital audio and/or visual content signal from said first device to a said remotely located device in compressed and optimized format and wherein said digital audio and/or visual content signal is compressed by one or more algorithms effective for compression and over the air delivery of said content and said digital audio and/or visual content is optimized by one or more algorithms effective for optimization of said content for said over the air wireless delivery.

2. The virtual wireless cable system of claim 1 wherein said system is implemented in place of a serial cable employing RS-232 standards and/or a USB cable.

3. The virtual wireless cable system of claim 1 employing RF cellular telephony system.

4. The virtual wireless cable system of claim 1 wherein said system provides for data transmission rates of a minimum of 9600 bit/s.

5. The virtual wireless cable system of claim 1 wherein said system transmits digital data signals in the form of data packets.

6. The virtual wireless cable system of claim 1 wherein said first device is a cell phone or a land line phone.

7. The virtual wireless cable system of claim 1 wherein said remote device is operated by voice recognition.

8. The virtual wireless cable system of claim 1 wherein said remotely located device is employed for access to a secured area.

9. The virtual wireless cable system of claim 1 wherein said system transmits a biometric data signals to a remotely controlled device for access to a secured area or information.

10. The virtual wireless cable system of claim 1 wherein said remotely controlled device is an automobile diagnostics system effective to provide automobile diagnostic information on demand.

11. The virtual wireless cable system of claim 1 wherein said remotely controlled device is a health care diagnostics system effective to provide human and/or animal health diagnostic information on demand.

12. The virtual wireless cable system of claim 1 wherein said remotely controlled device is a musical sound system.

13. The virtual wireless cable system of claim 1 wherein said remotely controlled device is a movie or video or television system.

14. The virtual wireless cable system of claim 1 wherein said remotely controlled device is a vehicle ignition, a watercraft ignition, a heating and/or air conditioning system for a vehicle or watercraft.

15. The virtual wireless cable system of claim 1 wherein said remotely controlled device is selected from window and door controls, telephone systems, computers, security cameras, security systems, intercom systems, driveway sensors, mailbox sensors, automated shutter, drape and curtain controls, automated pet doors and pet feeders, surge suppressors, lighting control, floodlights, automated sprinkling systems, automated pond feeders, microwaves, refrigeration systems, garage doors.

16. The virtual wireless cable system of claim 1 wherein said files device is a bar code reader device effective to place an order at a remote location by way of a remotely controlled device.
